(19) United States
(12) Patent Application Publication
(10) Pub. No.: US 2026/0063550 A1
(43) Pub. Date: Mar. 5, 2026

(54) SYSTEM AND METHOD FOR IN-LINE OPTICAL SENSING OF HYDROGEN PEROXIDE

(71) Applicant: CardioQuip, LLC, College Station, TX (US)

(72) Inventors: John DEAN, College Station, TX (US); William STEWART, College Station, TX (US); Douglas PLATT, College Station, TX (US); Jon Lowrey GARDNER, College Station, TX (US)

(21) Appl. No.: 19/383,323

(22) Filed: Nov. 7, 2025

Related U.S. Application Data
(62) Division of application No. 17/899,075, filed on Aug. 30, 2022, now abandoned.
(60) Provisional application No. 63/239,825, filed on Sep. 1, 2021.

(51) Int. Cl. G01N 21/59; G01N 21/33; G01N 33/18; G01N 21/31

(52) U.S. Cl. CPC G01N 21/59; G01N 21/33; G01N 33/1853; G01N 2021/3155; G01N 2201/062

(57) ABSTRACT

A water monitoring system comprising a hydrogen peroxide sensor configured to determine a concentration of hydrogen peroxide in water in a conduit. The hydrogen peroxide sensor further comprises an ultraviolet light sensor configured to determine an ultraviolet light absorbance level of the water in the conduit. The ultraviolet light absorbance level is used to determine the concentration of hydrogen peroxide. The hydrogen peroxide sensor may further comprise a visible light sensor configured to determine a turbidity level of the water in the conduit. The turbidity level also is used to determine the concentration of hydrogen peroxide.

### CROSS-REFERENCE TO RELATED APPLICATION(S) AND CLAIM OF PRIORITY

The present application is a divisional of U.S. Patent Application No. 17/899,075, filed August 30, 2022, which claims priority to U.S. Provisional Patent No. 63/239,825, filed September 1, 2021, entitled “Hydrogen Peroxide Sensor And Method Of Operation”. The entire contents of the above noted applications are incorporated by reference as part of the disclosure of this document.

### TECHNICAL FIELD

The present application relates generally to water systems and, more specifically, to an apparatus and method for monitoring real-time concentrations of hydrogen peroxide in liquids through absorbance readings.

### BACKGROUND

Protecting water resources poses a global challenge as drought conditions caused by climate change and pollution caused by industrial and agricultural runoff make accessing clean water an increasingly difficult task. An important strategy for coping with these problems is water conservation, which often requires treating and recirculating water in a wide variety of applications. As a result, water treatment systems have become ubiquitous in modern society. By way of example, water treatment systems are implemented in waste-water treatment facilities, food and beverage processing, pool water treatments, medical heating and cooling systems, and the like.

Large numbers of people access water through well-water systems, which typically bypass many water treatment systems in municipal water facilities. To decontaminate groundwater, users typically utilize chemical-injection systems in-line with their plumbing. These chemical injection systems often utilize hydrogen peroxide (H₂O₂) as the method for decontamination, as hydrogen peroxide naturally degrades to safe concentrations by the time it is ready for human use.

Medical perfusion systems may implement heater-cooler systems that regulate the temperature of a patient’s body through thermal transfer with a circulating liquid. For such systems, the Food and Drug Administration (FDA) requires medical device manufacturers to revalidate their cleaning and disinfection procedures to ensure that the water quality of their systems never exceeds unsafe levels of bacterial contamination. The industry has been shifting to specifying the perpetual use of water mixed with disinfectants, such as hydrogen peroxide, that can act as a mitigant for microbial growth.

There are a limited number of solutions for measuring the concentration of hydrogen peroxide in an aqueous solution. One method uses test strips that change color based on the hydrogen peroxide concentration. However, test strips are prone to user error when comparing the color of the test strip with a reference color strip. Test strips require a user to perform testing manually. Test strips are also disposable and produce waste. Devices are available to analyze the resultant color on the test strip. However, these devices are expensive and have poor sensitivities.

Another method uses amperometric sensors that detect hydrogen peroxide concentrations as hydrogen peroxide comes in contact with an active electrode and is oxidized on the surface. Amperometric sensors do not require user intervention to keep track of measurements. Therefore, they can provide live measurements of hydrogen peroxide concentrations and can be very sensitive to hydrogen peroxide concentrations. However, amperometric sensors are complex, require maintenance to replace a membrane, and must be calibrated periodically.

Still another method uses fluorescent optics, which involves reacting peroxide with an optically active membrane, called an optode, or a reagent in solution to increase the wavelength of a measured light source. Fluorescent sensors are capable of long-lasting automated measurements, but the optode must be replaced often, and the sensors are not resistant to pressure and temperature changes.

Therefore, there is a need for a hydrogen peroxide sensor that can monitor real-time concentrations of aqueous hydrogen peroxide. There is a need for a hydrogen peroxide sensor that does not require frequent maintenance of parts through its lifecycle and does not require user intervention to measure the hydrogen peroxide. There is a further need for a hydrogen peroxide sensor that does not require complicated periodic calibrations that the user must perform.

### SUMMARY

To address the above-discussed deficiencies of the prior art, it is a primary object of the present disclosure to provide a water monitoring system comprising a hydrogen peroxide sensor configured to determine a concentration of hydrogen peroxide in water in a conduit. The hydrogen peroxide sensor further comprises an ultraviolet light sensor configured to determine an ultraviolet light absorbance level of the water in the conduit. The ultraviolet light absorbance level is used to determine the concentration of hydrogen peroxide. The hydrogen peroxide sensor further comprises a visible light sensor configured to determine a turbidity level of the water in the conduit. The turbidity level also is used to determine the concentration of hydrogen peroxide.

It is another object of the present disclosure to provide a method of operating a water monitoring system comprising transferring water within the water treatment system via a conduit and determining using a hydrogen peroxide sensor a concentration of hydrogen peroxide in the water in the conduit. Determining the hydrogen peroxide concentration comprises determining an ultraviolet light absorbance level of the water in the conduit. Determining the hydrogen peroxide concentration further comprises determining a turbidity level of the water in the conduit.

It is still another object of the present disclosure to provide a water treatment system comprising a reservoir configured to store water, a heater-cooler system configured to regulate the temperature of the water stored in the reservoir, a conduit for transferring the water within the water treatment system, and a hydrogen peroxide sensor configured to determine a concentration of hydrogen peroxide in the water in the conduit. The hydrogen peroxide sensor comprises an ultraviolet light sensor configured to determine an ultraviolet light absorbance level of the water in the conduit, wherein the ultraviolet light absorbance level is used to determine the concentration of hydrogen peroxide. The hydrogen peroxide sensor further comprises a visible light sensor configured to determine a turbidity level of the water in the conduit, wherein the turbidity level also is used to determine the concentration of hydrogen peroxide.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a patient thermal regulation system according to one embodiment of the disclosure.

FIG. 2 illustrates an in-line hydrogen peroxide sensor according to an embodiment of the disclosure.

FIG. 3 illustrates the optical paths of the ultraviolet sensor and the visible light sensor according to an embodiment of the disclosure.

FIG. 4 is a flow diagram illustrating the operation of the in-line hydrogen peroxide sensor according to an embodiment of the disclosure.

FIG. 5 is a graph of example results of the in-line hydrogen peroxide sensor according to an embodiment of the disclosure.

### DETAILED DESCRIPTION

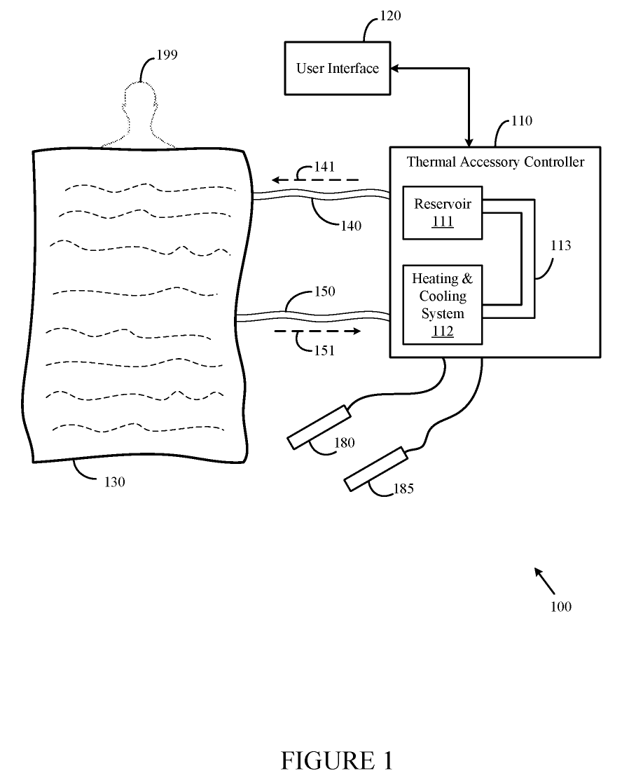

FIGS. 1 through 5, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged liquid treatment system that handles a liquid containing hydrogen peroxide.

The present disclosure describes a hydrogen peroxide absorbance sensor that is implemented as an in-line, or shunted, component that can be attached to any water path. Once attached to a water path, the hydrogen peroxide sensor is configured to monitor real-time concentrations of hydrogen peroxide in solution through absorbance readings. The disclosed hydrogen peroxide absorbance sensor is suitable for many applications, including monitoring hydrogen peroxide concentrations in cardiovascular heater-coolers, in process water disinfection, in waste-water treatment, in swimming pool water treatments, and the like.

FIG. 1 illustrates a patient thermal regulation system 100 according to one embodiment of the disclosure. The thermal management system may exist in the form of a heater-cooler device, blanket or pad warmer, or any other patient temperature management device. The patient thermal regulation system 100 comprises a thermal accessory controller 110, a user interface 120, a patient accessory 130, an output conduit 140, an input conduit 150, and electronic temperature probes 180 and 185. The user interface 120 allows a user or operator to control the operation of the thermal accessory controller 110. In an advantageous embodiment, the user interface 120 may be a laptop computer, a mobile phone, or a tablet device that communicates by wireline or wirelessly with the thermal accessory controller 110.

In an example embodiment, temperature probe 180 may be an oral thermometer and temperature probe 185 may be a rectal thermometer. The thermal accessory controller 110 reads temperature recordings from temperature probes 180 and 185 and, in response, may increase (heat) or decrease (cool) the temperature of a liquid that circulates through the patient accessory 130. In a heating mode, the warmed liquid provides thermal energy to the patient by contacting the patient accessory 130, and the patient accessory 130 contacting the patient 199. In a cooling mode, the cooled liquid absorbs thermal energy from the patient 199 through the patient accessory 130.

As indicated by the dotted directional arrow 141, the output conduit 140 (e.g., a hose 140) carries temperature-controlled liquid from the thermal accessory controller 110 to the patient accessory 130. As indicated by the dotted directional arrow 151, the input conduit 150 (e.g., a hose 150) returns the temperature-controlled liquid from the patient accessory 130 back to the thermal accessory controller 110. The returned liquid may then be rewarmed or cooled as needed.

In the example embodiment in FIG. 1, the patient accessory 130 comprises a blanket 130 that covers the body of a patient 199. However, this is by way of example only and should not be construed to limit the scope of the disclosure or the claims below. In alternate embodiments, the patient accessory 130 may comprise a pad 130 on which the patient 199 lies or a garment 130 that the patient 199 wears or a heat exchanger. For the sake of clarity and conciseness, the following descriptions shall assume that the patient accessory 130 comprises a thermal blanket 130.

The thermal accessory controller 110 comprises a reservoir 111, a heating and cooling system 112, and a conduit 113 that transfers the liquid from the reservoir 111 to the heating and cooling system 112. In an example embodiment, the liquid is an aqueous solution of water and hydrogen peroxide (H2O2). Another conduit (not shown) transfers the liquid from the heating and cooling system 112 back to the reservoir 111. Still other conduits (not shown) transfer the liquid from the internal plumbing to output conduit 140 and from the input conduit 150 back to the internal plumbing.

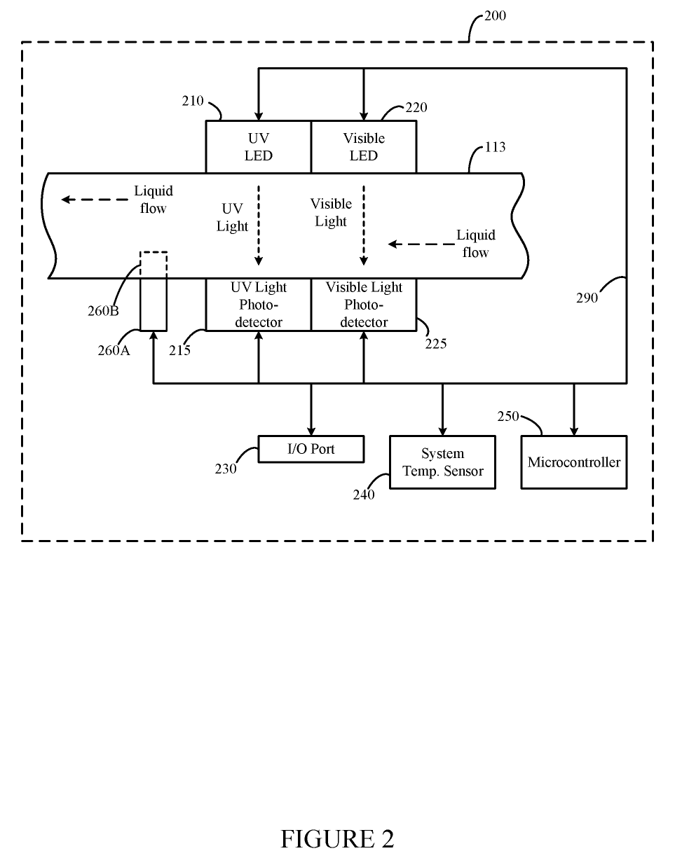

According to the principles of the present disclosure, the thermal accessory controller 110 further includes a hydrogen peroxide sensor (shown below in FIG. 2) that is configured to analyze the liquid as the liquid passes through, or reflects from within, the conduit 113. To accomplish this, a portion of the conduit 113 comprises a transparent aperture (e.g., quartz window). The hydrogen peroxide sensor includes an ultraviolet (UV) sensor and, potentially, a visible light sensor that transmit UV light and, potentially, visible light through the transparent aperture in order to determine the level of H2O2 in the liquid. In this way, the conduit 113 functions as a cuvette.

In the above-described embodiment, the hydrogen peroxide sensor is configured to analyze the liquid in conduit 113. However, this is by way of illustration only and should be not construed to limit the scope of the disclosure of the claim herein. In alternate embodiments, the hydrogen peroxide sensor may be attached to any water path, including, for example, the output conduit 140, the input conduit 150, or the other plumbing (e.g., tubing, pipes, etc.) within the thermal accessory controller 110.

FIG. 2 illustrates an in-line hydrogen peroxide sensor 200 according to an embodiment of the disclosure. The in-line hydrogen peroxide sensor 200 is configured to analyze a liquid flowing through the conduit 113. Dotted line arrows indicate the direction (right to left) of liquid flow in conduit 113. Hydrogen peroxide sensor 200 comprises an ultraviolet (UV) light-emitting diode (LED) 210, UV light photodetector 215, a visible light-emitting diode (LED) 220, and a visible light photodetector 225.

Collectively, the ultraviolet (UV) light-emitting diode (LED) 210 and the UV light photodetector 215 comprise an ultraviolet light sensor configured to determine the H2O2 concentration level in the liquid using UV absorbance calculations. Collectively, the visible light-emitting diode (LED) 220 and the visible light photodetector 225 comprise a turbidity light sensor configured to determine the turbidity level of the liquid in conduit 113.

Hydrogen peroxide sensor 200 also includes an input/output (I/O) port 230, a system temperature sensor 240, a microcontroller 250, a liquid temperature sensor 260, and a communications bus 290. In an example embodiment, liquid temperature sensor 260 may include a first portion 260A that is external to conduit 113 and a second portion 260B that is inserted into the conduit 113 and contacts the liquid in conduit 113.

At least a portion of conduit 113 is a transparent material, such as glass or quartz, to enable the UV LED 210 and the visible LED 220 to transmit (or emit) UV light and visible light, respectively, into conduit 113. Similarly, the transparent portion enables the UV light photodetector (PD) 215 to receive and to detect UV light emitted from conduit 113. The transparent portion further enables the visible light photodetector (PD) 225 to receive and detect visible light emitted from conduit 113.

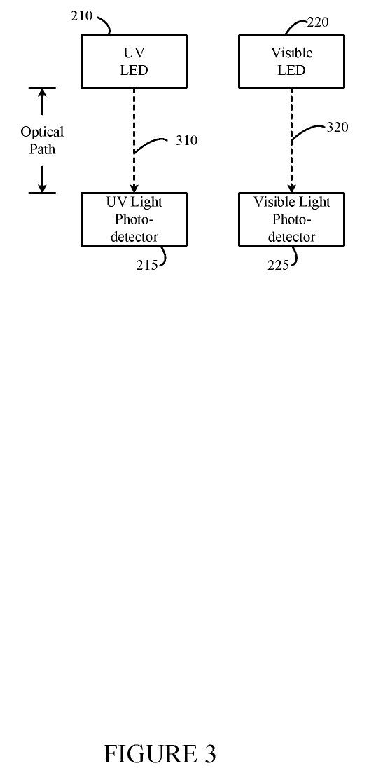

FIG. 3 illustrates the optical paths of the ultraviolet light sensor and the visible light sensor according to an embodiment of the disclosure. UV light emitted by UV LED 210 travels optical path 310 to UV light PD 215. Visible light emitted by visible LED 220 travels optical path 320 to visible light PD 225.

Returning to FIG. 2, microcontroller 250 controls the overall operation of hydrogen peroxide sensor 200. Microcontroller 250 communicates via bus 290, which may exist as a wired, wireless, or optical channel, with UV LED 210 to activate UV LED 210 and cause UV LED 210 to transmit UV light into conduit 113. Microcontroller 250 communicates with UV light photodetector (PD) 215 to detect the transmitted UV light from the UV LED 210 in conduit 113. Microcontroller 250 similarly communicates via bus 290 with visible LED 220 to activate visible LED 220 and cause visible LED 220 to transmit visible light into conduit 113. Microcontroller 250 communicates with visible light photodetector (PD) 225 to detect the transmitted visible light from the visible LED 220 in conduit 113.

Microcontroller 250 is further configured to cause system temperature sensor 240 to record periodically the ambient temperature of the electronics in the thermal accessory controller 110. Microcontroller 250 also causes liquid temperature sensor 260 to record periodically the temperature of the liquid in conduit 113.

According to the principles of the present disclosure, in-line hydrogen peroxide sensor 200 may be attached to any water path in order to monitor real-time concentrations of hydrogen peroxide in solution through absorbance readings. There are many applications for sensor 200, including monitoring hydrogen peroxide concentrations of cardiovascular heater-coolers, process water disinfection, waste-water treatment, swimming pool water treatments, and the like.

In an example embodiment, hydrogen peroxide sensor 200 may detect hydrogen peroxide concentrations using optical means via absorbance readings in a particular UV range (190-300 nm range). The ratio of absorbed light detected by UV light photodetector 215 to emitted light transmitted by UV LED 210 may be used to correlate the concentration of peroxide to absorbance. This may be done using variations of the Beer-Lambert Law.

Microcontroller 250 may use I/O port 230 to transmit notification to users that the hydrogen peroxide concentrations are out of range. Microcontroller 250 may notify user via a visual and/or audible alarm that the water in the water treatment device is not meeting the recommended concentration of hydrogen peroxide for safe use. If the hydrogen peroxide in a heater-cooler system is below the specified hydrogen peroxide concentration for a certain amount of time, microcontroller 250 may prompt the user to perform a cleaning and disinfection procedure. This ensures that any micro-organisms that could have grown during this time period are killed before the hydrogen peroxide concentration is reestablished to its specified levels.

According to the principles of the present disclosure, microcontroller 250 in hydrogen peroxide sensor 200 may use variations of the Beer-Lambert law or absorbance measurement ratios to correlate the concentration of peroxide in solution to the absorbance at a specified wavelength. By emitting a known intensity of light through the liquid in conduit 113, absorbance may be determined by analyzing the resulting light at the UV photodetector 215. Hydrogen peroxide has strong absorbance in the far-UV (190-260 nm) range, which means that its concentration can be analyzed by emitting light at these wavelengths to gather its absorbance data. The operation of the hydrogen peroxide sensor 200 can be further improved by using visible LED 220 and visible light photodetector 225 as a visible-spectrum turbidity sensor that is configured to determine if the water quality is good enough for the hydrogen peroxide sensor 200 to be accurate. This turbidity sensor may impact the calculations used, depending on water quality.

In an example embodiment, the hydrogen peroxide sensor 200 may use a UV LED 210 with a wavelength output between 190-300 nm and a UV photodiode sensitive to UV light in the 190-300 nm range. The turbidity optical sensor may use a visible-range LED with a wavelength output between 380-700 nm and a visible light photodiode sensitive to visible light in the 380-700 nm range. Other embodiments may utilize other wavelength ranges for chemical or biological sensing.

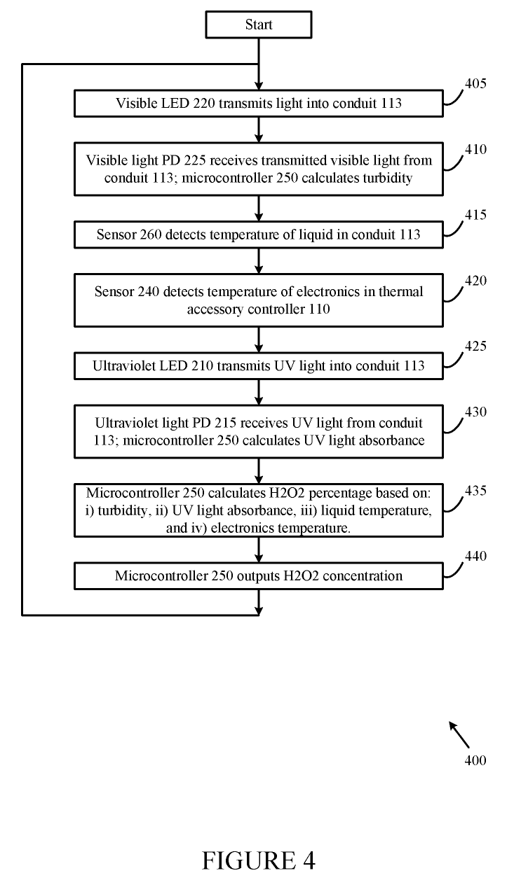

FIG. 4 is a flow diagram illustrating the operation of the in-line hydrogen peroxide sensor 200 according to an embodiment of the disclosure. In 405, the visible LED 220 transmits (or emits) visible light into conduit 113 (or conduits 140 and 150). In 410, the visible light photodetector (PD) 225 receives the transmitted visible light from conduit 113 and microcontroller 250 calculates the turbidity level of the liquid in conduit 113 based on the level of received visible light. In 415, temperature sensor 260 detects the temperature of the liquid in conduit 113. In 420, temperature sensor 240 detects the ambient temperature of the electronics in thermal accessory controller 110. In 425, ultraviolet (UV) LED 210 transmits (or emits) UV light into conduit 113. In 430, ultraviolet light PD 215 receives UV light from conduit 113 and microcontroller 250 calculates the UV light absorbance level.

In 435, microcontroller 250 calculates the hydrogen peroxide (H2O2) percentage based on: i) the turbidity level, ii) the UV light absorbance level , iii) the liquid temperature, and iv) the electronics temperature. Finally, in 440, microcontroller 250 outputs the H2O2 concentration to the user interface 120 via I/O port 230.

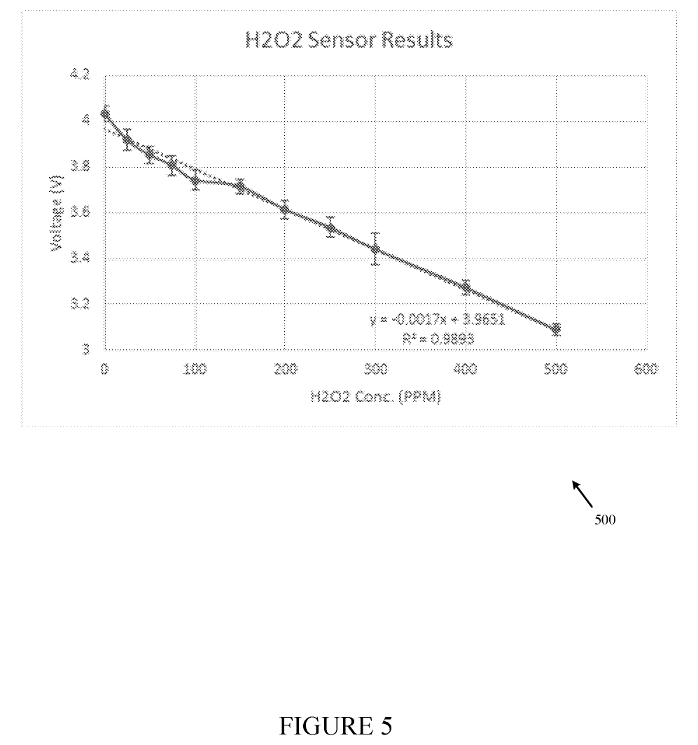

FIG. 5 is a graph 500 of example results of the in-line hydrogen peroxide sensor 300 according to an embodiment of the disclosure. The horizontal axis of graph 500 shows the H2O2 concentration in parts-per-million (PPM). The vertical axis of graph 500 shows example voltage readouts from the UV light photodetector 215.

Although the present disclosure has been described with an exemplary embodiment, various changes and modifications may be suggested to one skilled in the art. It is intended that the present disclosure encompass such changes and modifications as fall within the scope of the appended claims.

WHAT IS CLAIMED IS:

1. A water monitoring system comprising:
a conduit through which liquid is cycled through a patient accessory and a thermal accessory controller,
wherein the patient accessory is external to but fluidly connected to the thermal accessory controller;
wherein the water monitoring system is a part of the thermal accessory controller;
a hydrogen peroxide sensor that is configured to determine an ultraviolet light absorbance level of the liquid flowing along the conduit that comprises:
an ultraviolet light-emitting diode configured to transmit ultraviolet light through a first transparent aperture of the conduit into the liquid flowing along the conduit; and
an ultraviolet light photodetector configured to receive and to measure, through a second transparent aperture of the conduit, the ultraviolet light transmitted by the ultraviolet light-emitting diode into the liquid flowing along the conduit,
wherein the ultraviolet light-emitting diode and the ultraviolet light photodetector are disposed on opposing sides of the conduit;
a microcontroller that is configured to determine a concentration of hydrogen peroxide in the liquid flowing along the conduit based on an analysis of the ultraviolet light absorbance level; and
an alarm that is configured to indicate that the concentration of hydrogen peroxide in the liquid flowing along the conduit exceeds a threshold in response to receiving, from the microcontroller, an indication that the concentration of hydrogen peroxide in the liquid flowing along the conduit exceeds the threshold.

2. The water monitoring system of claim 1, wherein the hydrogen peroxide sensor further comprises:
a visible light sensor configured to identify a turbidity level of the liquid flowing along the conduit.

3. The water monitoring system of claim 2, wherein the visible light sensor comprises:
a visible light-emitting diode configured to transmit visible light through the first transparent aperture of the conduit into the liquid flowing along the conduit; and
a visible light photodetector configured to receive and to measure, through the second transparent aperture of the conduit, the visible light transmitted by the visible light-emitting diode into the liquid flowing along the conduit.

4. The water monitoring system of claim 1, wherein the hydrogen peroxide sensor further comprises:
a first temperature sensor configured to determine an ambient temperature of an electronic component of the water monitoring system.

5. The water monitoring system of claim 1, wherein the hydrogen peroxide sensor further comprises:
a second temperature sensor configured to determine a temperature of the liquid flowing along the conduit,
wherein the second temperature sensor is positioned such that at least a portion of the second temperature sensor is inserted into the conduit.

6. The water monitoring system of claim 1, wherein the alarm indicates that the concentration of hydrogen peroxide in the liquid flowing along the conduit exceeds the threshold via a visual alert, an audible alert, or a visual and audible alert.

7. The water monitoring system of claim 1, wherein the microcontroller is configured to determine the concentration of hydrogen peroxide in the liquid flowing along the conduit based on one or more of the ultraviolet light absorbance level, a turbidity level, an ambient temperature of an electronic component of the water monitoring system, and a temperature of the liquid flowing along the conduit.

8. The water monitoring system of claim 1, wherein the liquid flowing along the conduit is temperature controlled by the thermal accessory controller.

9. A method performed by a water monitoring system, the method comprising:
transferring liquid within the water monitoring system via a conduit;
identifying an ultraviolet light absorbance level of the liquid flowing along the conduit using a hydrogen peroxide sensor,
wherein the hydrogen peroxide sensor comprises an ultraviolet light-emitting diode and an ultraviolet light photodetector that are disposed on opposing sides of the conduit;
determining a concentration of hydrogen peroxide in the liquid flowing along the conduit based on the identified ultraviolet light absorbance level; and
causing an alert that indicates that the concentration of hydrogen peroxide in the liquid flowing along the conduit exceeds a particular threshold in response to determining that the concentration of hydrogen peroxide in the liquid flowing along the conduit exceeds the particular threshold.

10. The method of claim 9, wherein the ultraviolet light-emitting diode transmits ultraviolet light into the liquid flowing along the conduit, and wherein the ultraviolet light photodetector receives and measures the ultraviolet light transmitted by the ultraviolet light-emitting diode into the liquid flowing along the conduit.

11. The method of claim 9, further comprising:
identifying a turbidity level of the liquid flowing along the conduit using the hydrogen peroxide sensor, the hydrogen peroxide sensor further comprising:
a visible light-emitting diode configured to transmit visible light into the liquid flowing along the conduit; and
a visible light photodetector configured to receive and to measure the visible light transmitted by the visible light-emitting diode into the liquid flowing along the conduit.

12. The method of claim 9, further comprising:
identifying an ambient temperature of the water monitoring system using the hydrogen peroxide sensor, the hydrogen peroxide sensor further comprising a first temperature sensor.

13. The method of claim 9, further comprising:
identifying a liquid temperature of the liquid flowing along the conduit using the hydrogen peroxide sensor, the hydrogen peroxide sensor further comprising:
a second temperature sensor, the second temperature sensor positioned such that at least a portion of the second temperature sensor is inserted into the conduit to identify the liquid temperature of the liquid flowing along the conduit.

14. The method of claim 9, wherein the alert comprises a visual, audible, or visual and audible alert.

15. The method of claim 9, further comprising:
identifying the ultraviolet light absorbance level of the liquid flowing along the conduit using the hydrogen peroxide sensor, a turbidity level of the liquid flowing along the conduit, an ambient temperature of the water monitoring system, and a liquid temperature of the liquid flowing along the conduit; and
determining the concentration of hydrogen peroxide in the liquid flowing along the conduit based on or more of the ultraviolet light absorbance level, the turbidity level, the ambient temperature, and the liquid temperature.

16. A thermal accessory controller comprising:
a conduit configured to move water through the thermal accessory controller;
a hydrogen peroxide sensor affixed to the conduit, the hydrogen peroxide sensor comprising:
an ultraviolet light sensor configured to determine an ultraviolet light absorbance level of the water moving through the thermal accessory controller;
a microcontroller configured to determine a concentration of hydrogen peroxide in the water moving through the thermal accessory controller based on the ultraviolet light absorbance level; and
an alarm, the alarm configured to indicate that the concentration of hydrogen peroxide in the water moving through the thermal accessory controller exceeds a particular threshold.

17. The thermal accessory controller of claim 16, wherein the ultraviolet light sensor further comprises:
an ultraviolet light-emitting diode that transmits ultraviolet light into the water moving through the thermal accessory controller; and
an ultraviolet light photodetector that receives and measures the ultraviolet light transmitted by the ultraviolet light-emitting diode into the water moving through the thermal accessory controller.

18. The thermal accessory controller of claim 16, wherein the hydrogen peroxide sensor further comprises:
a visible light sensor that is configured to identify a turbidity level of the water moving through the thermal accessory controller, the visible light sensor comprising:
a visible light-emitting diode configured to transmit visible light through a first transparent aperture of the conduit into the water moving through the thermal accessory controller; and
a visible light photodetector configured to receive and to measure, through a second transparent aperture of the conduit, the visible light transmitted by the visible light-emitting diode into the water moving through the thermal accessory controller.

19. The thermal accessory controller of claim 16, wherein the alarm is configured to indicate that the concentration of hydrogen peroxide in the water moving through the thermal accessory controller exceeds the particular threshold by a visual alert, an audible, or a visual and audible alert.

20. The thermal accessory controller of claim 16, wherein the hydrogen peroxide sensor further comprises:
a first temperature sensor configured to determine an ambient temperature of an electronic component of the thermal accessory controller; and
a second temperature sensor configured to determine a temperature of the water moving through the thermal accessory controller,
wherein the second temperature sensor is positioned such that at least a portion of the second temperature sensor is inserted into the conduit.
